(19) United States
(12) Patent Application Publication
(10) Pub. No.: US 2025/0087819 A1
(43) Pub. Date: Mar. 13, 2025

(54) ELECTROCHEMICAL DEVICE, SENSOR, AND SENSOR SYSTEM

(71) Applicant: KABUSHIKI KAISHA TOSHIBA, Tokyo (JP)

(72) Inventors: Akira FUJIMOTO, Kawasaki Kanagawa (JP); Yosuke AKIMOTO, Yokohama Kanagawa (JP); Hiroaki YAMAZAKI, Yokohama Kanagawa (JP)

(73) Assignee: KABUSHIKI KAISHA TOSHIBA, Tokyo (JP)

(21) Appl. No.: 18/782,083

(22) Filed: Jul. 24, 2024

(30) Foreign Application Priority Data
Sep. 13, 2023 (JP) 2023-148425

(51) Int. Cl. H01M 50/293; G01N 27/407; G01N 33/00

(52) U.S. Cl. CPC H01M 50/293; G01N 27/4074; G01N 33/0036

(57) ABSTRACT

An electrochemical device includes a housing, a first electrochemical element present inside the housing and a porous structure occupying at least a part of the inside of the housing.

### CROSS-REFERENCE TO RELATED APPLICATIONS

This application is based upon and claims the benefit of priority from Japanese Patent Application No. 2023-148425 filed on Sep. 13, 2023, and the entire contents of which are incorporated herein by reference.

### FIELD

Embodiments described herein relate generally to an electrochemical device, a sensor, and a sensor system.

### BACKGROUND

For example, there is a sensor that senses oxygen and hydrogen.

In such a sensor, generally, when the sensor is exposed to vapor, gas, and the like in the atmosphere in which the sensor is installed, a sensing function is deteriorated.

### BRIEF DESCRIPTION OF THE DRAWINGS

FIG. 1 is a schematic cross-sectional view illustrating an electrochemical device according to an embodiment.

FIG. 2 is a schematic cross-sectional view illustrating another example of the electrochemical device according to the embodiment.

FIG. 3 is a schematic cross-sectional view illustrating still another example of the electrochemical device according to the embodiment.

FIG. 4 is a schematic view illustrating an operation of the electrochemical device.

FIG. 5 is a schematic cross-sectional view illustrating a sensor according to the embodiment.

FIGS. 6A to 6C are schematic plan views and a schematic cross-sectional view illustrating a substrate included in the sensor according to the embodiment.

FIGS. 7A and 7B are schematic cross-sectional views illustrating a first film that can be included in the sensor according to the embodiment.

FIG. 8 is a schematic cross-sectional view illustrating another example of the sensor according to the embodiment.

### DETAILED DESCRIPTION

Hereinafter, embodiments will be described with reference to the drawings.

In the following description, components that exhibit the same or similar functions are denoted by the same reference numerals throughout all the drawings, and redundant description will be omitted.

Note that each drawing is a schematic view for promoting the description of an embodiment and the understanding thereof, and points regarding the shape, size, ratio, and the like may be different from those of the actual device, but the design of these points can be appropriately modified in consideration of the following description and known techniques.

### First Embodiment

A sensor that senses oxygen or hydrogen may be contaminated, thereby deteriorating a sensing function or causing a sensor circuit included in the sensor to fail. The sensor may be contaminated by exposure to moisture, gas, and the like in an atmosphere in which an electrochemical device is provided. Among the contaminations, for example, in order to suppress the contamination of the sensor by moisture (hereinafter, referred to collectively as water vapor, but an aspect may be liquid or gas), the electrochemical device includes an electrochemical element such as a dehumidifier.

In addition, the housing of the electrochemical device originally contains water vapor, and the water vapor contained in the housing comes out into the housing due to generation of heat or the like by driving of the sensor. As a result, the sensor and the sensor circuit provided inside the housing are exposed to water vapor for a long time, a failure of the sensor circuit occurs and a sensing function of the sensor deteriorate. Therefore, it is necessary to suppress exposure of the sensor and the sensor circuit to water vapor for a long time, to suppress deterioration of the sensing function of the sensor, and to suppress failure of the sensor circuit of the sensor.

Therefore, according to a first embodiment, there is provided an electrochemical device including a housing, a first electrochemical element present inside the housing, and a porous structure occupying at least a part of the inside of the housing.

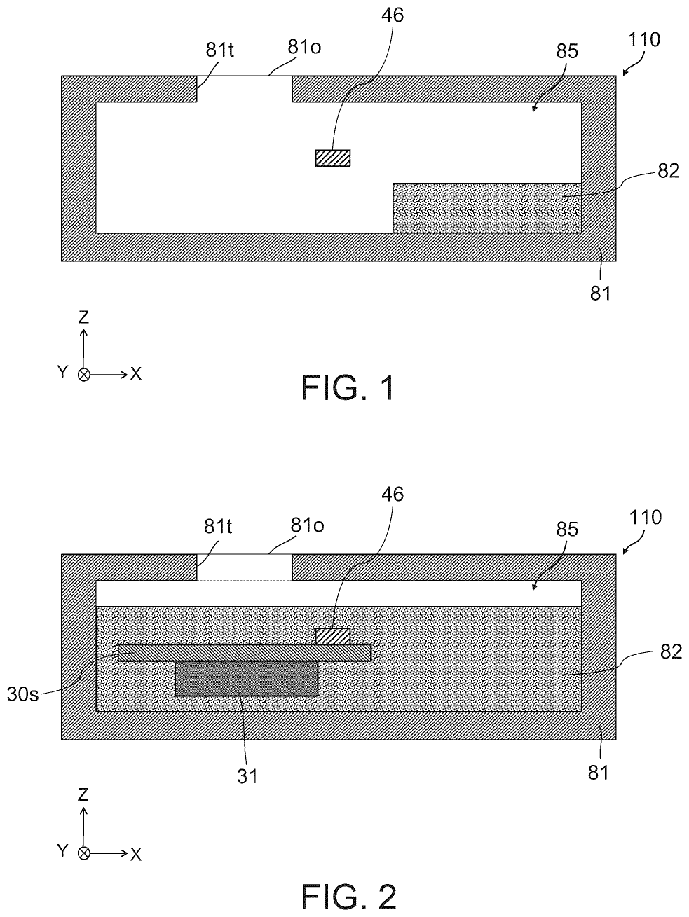

Hereinafter, the electrochemical device that suppresses deterioration of the sensing function in the sensor will be described with reference to the drawings. FIG. 1 is a schematic cross-sectional view illustrating the electrochemical device according to the first embodiment. In FIG. 1, an electrochemical device 110 includes a housing 81, a first electrochemical element 46 present inside the housing 81, and a porous structure 82 occupying at least a part of the inside of the housing 81. The housing 81 has an opening 81o and a through hole 81t. The opening 81o corresponds to an inlet or an outlet of the through hole 81t. The shape of the opening may be substantially quadrangular or circular, and the shape is not limited. In FIG. 1, a normal direction of the opening 81o is defined as a Z-axis direction, a direction perpendicular to the Z-axis direction is defined as an X-axis direction, and a direction perpendicular to the Z-axis and X-axis directions is defined as a Y-axis direction.

The porous structure 82 occupies at least a part of the inside of the housing 81. As a result, in a case where the humidity inside the housing 81 is kept lower than the humidity outside the housing 81, for example, in a case where the relative humidity inside the housing 81 is 60% or less, the porous structure 82 can take in water vapor entering the housing 81. The presence of the porous structure 82 having a large surface area inside the housing 81 makes it possible to increase the effective volume inside the housing 81, thereby making it possible to relatively reduce the humidity inside the housing 81.

The porous structure 82 preferably occupies at least a part of the inner wall of the housing 81. As such, the porous structure 82 can take in water vapor originally contained in the housing 81. This makes it possible to suppress exposure of the first electrochemical element 46 to water vapor for a long time. More preferably, the porous structure 82 covers the entire inner wall of the housing 81.

The filling rate of the porous structure 82 inside the housing 81 excluding the first electrochemical element 46 provided inside the housing 81 is preferably 10% or more and 100% or less.

The filling rate of porous structure 82 inside the housing 81 in another example of the electrochemical device according to the first embodiment described later is preferably 10% or more and 100% or less similarly. At this time, various components inside the housing 81 may be removed from the volume of the housing 81. That is, the filling rate to be obtained is a ratio of the porous structure to the volume inside the housing 81 excluding various components inside the housing 81.

FIG. 2 is a schematic cross-sectional view illustrating another example of the electrochemical device according to the first embodiment. In FIG. 2, the electrochemical device 110 further includes a substrate 30s and a first battery 31, the first electrochemical element 46 is provided on the substrate 30s, and the first battery 31 is provided on a surface on the substrate 30s opposite to a surface on which the first electrochemical element 46 is provided.

The first battery 31 is provided on a surface of the substrate 30s on which a microcomputer circuit is provided. A sensor circuit is provided on the surface on which the first electrochemical element 46 is provided, the surface being opposite to the surface of the substrate 30s on which the microcomputer circuit is provided. Note that the microcomputer circuit may be provided on the upper side, and the sensor circuit may be provided on the lower side.

The substrate 30s may contain, for example, a resin such as an epoxy resin, a phenol resin, a polyimide resin, or a glass epoxy resin including a glass cloth. Here, as described above, the substrate 30s is a resin substrate and originally contains water vapor contained in air. Water vapor originally contained in the substrate 30s is released from the substrate by heat generated by driving of the sensor circuit, the microcomputer circuit, and the like, and thus contaminates the circuit, the sensor, and the like described above. Therefore, as shown in FIG. 2, the porous structure 82 is preferably provided inside the housing 81 so as to take in water vapor that may be generated on the substrate 30s.

For example, the porous structure 82 is desirably configured to substantially cover the first battery 31, the substrate 30s, and the first electrochemical element 46.

The porous structure 82 may also be configured to substantially cover the first battery 31.

The porous structure 82 may also be configured to substantially cover the first battery 31 and to cover a part of the substrate 30s.

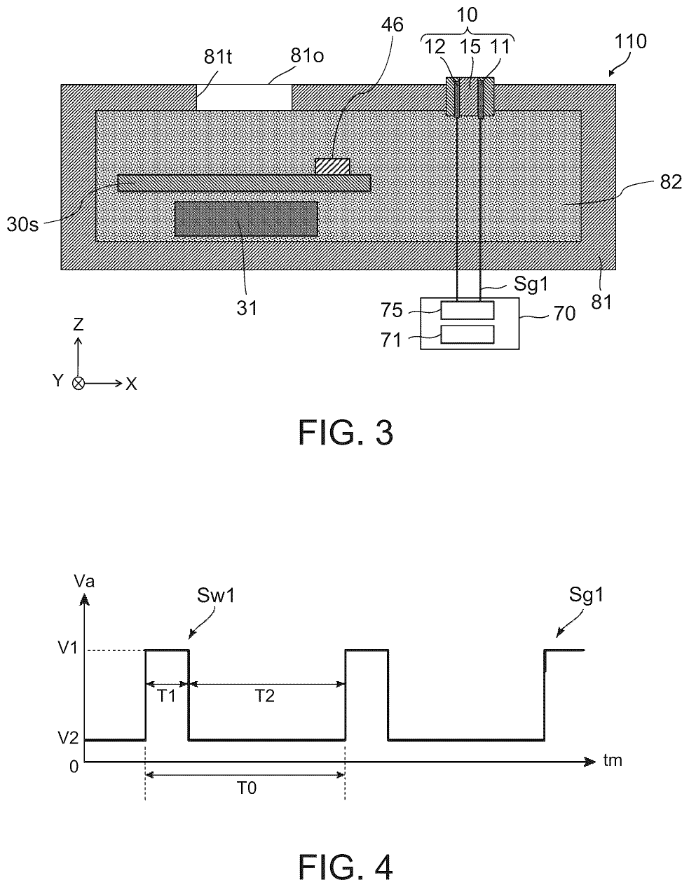

FIG. 3 is a schematic cross-sectional view illustrating still another example of the electrochemical device according to the first embodiment. In FIG. 3, the porous structure 82 is present between the substrate 30s and the first battery 31. The porous structure 82 is preferably present between the substrate 30s and the first battery 31.

On the surface of a substrate 31s on which the first battery 31 is provided, in addition to the heat generated from the driving of the sensor circuit, the microcomputer circuit, and the like, the heat due to the operation of the first battery 31 is transferred. As a result, more water vapor is released from the surface of the substrate 31s on which the first battery 31 is provided as compared with the surface of the substrate 30s on which the first electrochemical element 46 is provided. Therefore, with the configuration as shown in FIG. 3, the porous structure 82 covers the entire substrate 30s, and the water vapor released from the substrate 30s can be more quickly captured by the porous structure 82, so that an increase in humidity in the periphery can be suppressed.

In addition, since the porous structure 82 is present between the substrate 30s and the first battery 31, the first battery 31 is not in physical contact with the substrate 30s, it is possible to suppress transfer of heat to the substrate 30s due to driving of the first battery 31. This makes it possible to suppress the release of water vapor from the substrate 30s due to an increase in the temperature of the substrate 30s.

In addition, the electrochemical device 110 of FIG. 3 has a second electrochemical element 10. The second electrochemical element 10 includes a first electrode 11, a second electrode 12, and a member 15. The member 15 is provided between the first electrode 11 and the second electrode 12. For example, the member 15 contains a polymer electrolyte.

The second electrochemical element 10 can perform at least one of, for example, dehumidification, humidification, ozone generation, oxygen generation, oxygen removal, and hydrogen generation. By applying a voltage between the first electrode 11 and the second electrode 12, at least one of dehumidification, humidification, ozone generation, oxygen generation, oxygen removal, and hydrogen generation is possible.

The porous structure 82 provided inside the housing 81 preferably covers at least a part of the second electrochemical element 10. As a part of the second electrochemical element 10 is covered with the porous structure 82, entry of water vapor from the outside to the inside of the housing 81 through the member 15 can be prevented. More preferably, the entire portion present inside the housing 81 of the second electrochemical element 10 is covered with the porous structure 82.

Hereinafter, an example in which the second electrochemical element 10 has a dehumidifying function will be described. Dehumidification is performed by applying the voltage between the first electrode 11 and the second electrode 12. For example, when the voltage is applied, moisture in the housing 81 is separated into hydrogen and oxygen by electrochemical decomposition, and released to the outside of the housing 81 through the member 15. Accordingly, dehumidification is performed. The application of the voltage is performed, for example, by providing a battery.

For example, a voltage based on the second electrode 12 is applied to the first electrode 11. For example, when a voltage having a first polarity is applied to the first electrode 11, the second electrochemical element 10 releases water to the outside of the housing 81 through the member 15. Hereinafter, the first polarity is positive. Dehumidification is performed when the voltage having the first polarity is applied to the first electrode 11.

The second electrochemical element 10 can release water in a space 85 in the housing 81 to the outside of the housing 81 through the member 15. As a result, the space in the housing 81 is dehumidified.

The electrochemical device 110 can further include a controller 70. The controller 70 is electrically connected to the first electrode 11 and the second electrode 12. In this example, the controller 70 includes a circuit unit 75 and a second battery 71. The second battery 71 can supply power to the circuit unit 75. The circuit unit 75 can apply the voltage between the first electrode 11 and the second electrode 12. The voltage is, for example, a first signal Sg1. When the first signal Sg1 (voltage) is applied to the first electrode 11 and the second electrode 12, the electrochemical action (for example, dehumidification) in the second electrochemical element 10 is performed.

In the embodiment, the second electrochemical element 10 is driven by the second battery 71. This makes it possible to operate in a place where commercial power or the like is not supplied, and an application of the electrochemical device 110 and various devices using the electrochemical device 110 is expanded. Since a capacity of the second battery 71 is fixed, it is desired to reduce power consumption in the electrochemical device 110 in order to prolong a driving time of the electrochemical device 110.

In general, the second electrochemical element 10 is often driven by a DC signal (DC voltage) having a constant value. In this case, a DC current having a constant value flows in the second electrochemical element 10. As a result, power is always consumed, thereby increasing power consumption.

In the embodiment, the voltage (first signal Sg1) supplied to the second electrochemical element 10 is a duty signal. At this time, by setting the first signal Sg1 to a special waveform, it is possible to reduce power consumption while maintaining a high electrochemical action. A reason will be described later with reference to FIG. 4.

In the embodiment, the second electrochemical element 10 includes a cathode and an anode. The cathode is one of the first electrode 11 and the second electrode 12. The anode is the other of the first electrode 11 and the second electrode 12.

For example, the cathode may include a cathode base and a cathode side catalyst member provided on a surface of the cathode base. The anode may include an anode base and an anode side catalyst member provided on a surface of the anode base. At least a part of a solid polymer electrolyte film is provided between the cathode side catalyst member and the anode side catalyst member. The solid polymer electrolyte film corresponds to the member 15.

For example, the cathode base includes a carbon film (for example, carbon paper). The cathode side catalyst member contains carbon powder. Platinum adheres to a surface of the carbon powder. The carbon powder holds the platinum. For example, the anode base includes a titanium mesh. A platinum film is provided on a surface of the titanium mesh. The platinum film is formed by, for example, plating. The anode side catalyst member contains platinum particles and a fluororesin. The solid polymer electrolyte film contains the fluororesin. The fluororesin is, for example, a copolymer of a sulfonated tetrafluoroethylene-based fluororesin.

Next, an example of the first signal Sg1 will be described with reference to FIG. 4. FIG. 4 illustrates the first signal Sg1. The horizontal axis in FIG. 4 represents a time tm. The vertical axis represents a voltage Va between the first electrode 11 and the second electrode 12. The voltage Va is a potential based on a potential of the second electrode 12.

As illustrated in FIG. 4, the first signal Sg1 includes a waveform Sw1 that repeats in a first period TO. The waveform Sw1 includes a first period T1 and a second period T2. In the first period T1, the voltage Va is a first voltage V1 of the first polarity. In the second period T2, the voltage Va is a second voltage V2 of the first polarity. The first polarity is either positive or negative. In this example, the first polarity is positive. The first voltage V1 and the second voltage V2 are positive. An absolute value of the second voltage V2 is smaller than an absolute value of the first voltage V1.

In one example, the first voltage V1 is 2.5 V or more and 3.5 V or less. In one example, the second voltage V2 is 0.5 V or more and 1.5 V or less. As described above, since the second voltage V2 of the low voltage is also the first polarity (positive) and is not 0 voltage, a current flowing in a transition period from the first period T1 of the high voltage (first voltage V1) to the second period T2 of the low voltage (second voltage V2) hardly becomes a negative current. The current flowing in the transition period described above is a positive current, and even if the current flowing in the transition period is negative, the absolute value thereof is small. As a result, a reverse reaction in the electrochemical action, that is, a reaction in which moisture is released from the member 15 into the housing 81, is suppressed. In addition, by providing the second period T2 of the low-voltage second voltage V2, it is possible to reduce power consumption while maintaining the intended high electrochemical action (for example, dehumidification) in such a first signal Sg1.

The porous structure is preferably at least one selected from the group consisting of zeolite, silica gel, activated alumina, activated carbon, and porous polymer, but is not limited to the above-described materials as long as it can increase the effective volume inside the housing. The zeolite is a microporous crystalline aluminosilicate.

The housing is, for example, a resin. The housing may include zeolite, activated carbon, and silica gel.

The electrochemical device according to the first embodiment includes the housing, the first electrochemical element present inside the housing, and the porous structure occupying at least a part of the inside of the housing. This makes it possible to suppress deterioration of the sensing function.

### Second Embodiment

According to a second embodiment, a sensor including the electrochemical device according to the first embodiment and a detector provided inside the housing is provided.

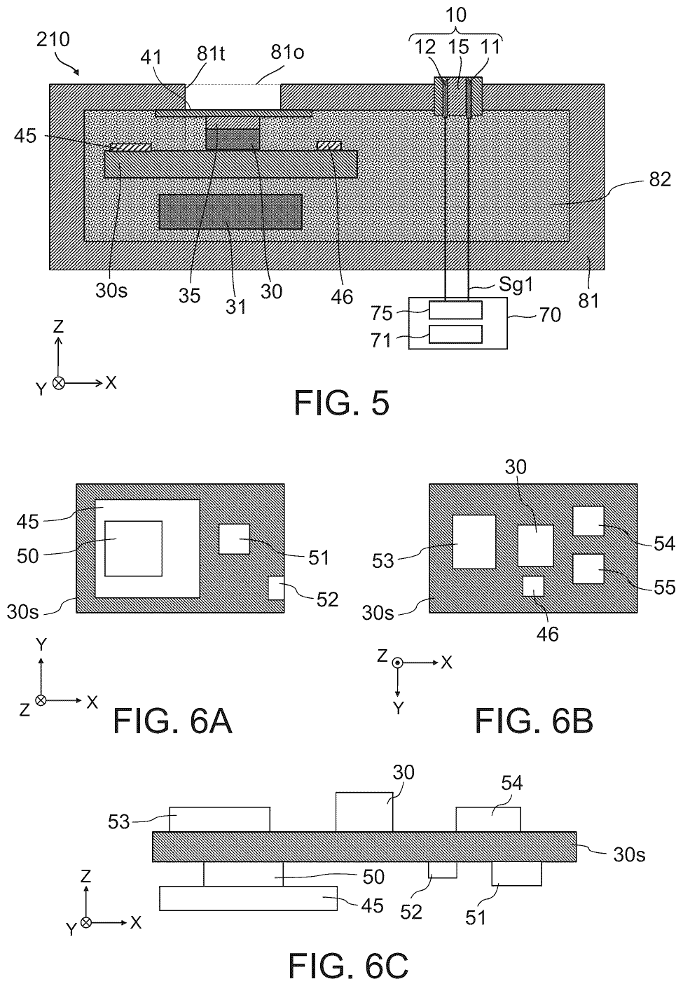

FIG. 5 is a schematic cross-sectional view illustrating the sensor according to the second embodiment. As illustrated in FIG. 5, a sensor 210 according to the embodiment includes the electrochemical device 110 according to the first embodiment and a detector 30. Furthermore, the sensor 210 may further include a communication unit 45. The detector 30 and the communication unit 45 included in the sensor 210 will be described later with reference to FIGS. 6A to 6C which are plan views and a cross-sectional view of the substrate 30s. The communication unit 45 is, for example, a communication circuit.

In the embodiment, the distance between the second electrochemical element 10 and the detector 30 is preferably short. For example, the detector 30 is preferably fixed the near the second electrochemical element 10. The distance between the second electrochemical element 10 and the detector 30 is, for example, 1 mm or more and 50 mm or less.

In FIG. 5, the sensor 210 includes a first film 41. The first film 41 is provided between the detector 30 and the first opening 81o of the housing 81. The first film 41 is, for example, provided so as to cover the opening 81o. At least a part of the first film 41 is porous. The first film 41 contains, for example, a resin containing fluorine. The first film 41 contains, for example, polytetrafluoroethylene (PTFE). For example, the first film 41 does not allow a liquid (such as water) to permeate therethrough. The first film 41 allows a gas (for example, hydrogen) to be detected by the detector 30 to permeate therethrough.

In the embodiment, the first film 41 may be in contact with the detector 30. Alternatively, the distance between the first film 41 and the detector 30 is 1 cm or less. By providing the detector 30 near the first film 41, the detection target gas that has passed through the first film 41 can be detected with higher accuracy.

By providing the first film 41 in the opening 81o, an influence of external humidity is suppressed in the space 85 in the housing 81. By an electrochemical action (for example, dehumidification) by the second electrochemical element 10, the state of the space 85 becomes an intended state. As will be described later, by providing a detector or the like in the housing 81, the detector can be maintained in an intended state (for example, low humidity).

As illustrated in FIG. 5, the sensor 210 may include the first battery 31. The first battery 31 can supply power to the detector 30. By providing the first battery 31, for example, a detection target can be detected even in a place where commercial power is not supplied.

In the sensor 210, the detector 30 is provided on the substrate 30s. In the sensor 210, the detector 30 is provided between the opening 81o and the substrate 30s. A lid 35 may be provided between the detector 30 and the first film 41. The lid 35 may have a hole. A humidity sensor 46 may be provided on the substrate 30s. The humidity sensor 46 may monitor humidity in the space 85 inside the housing 81.

FIGS. 6A to 6B are schematic plan views and a schematic cross-sectional view illustrating a substrate included in the electrochemical device according to the first embodiment. FIG. 6A is a plan view of a surface of the substrate on which the microcomputer circuit is provided. FIG. 6B is a plan view of a surface of the substrate on which the sensor circuit is provided. FIG. 6C is a cross-sectional view when cut in a direction intersecting the surface of the substrate.

In FIGS. 6A to 6C, a direction from a microcomputer circuit surface of the substrate 30s toward a sensor circuit surface is defined as a Z-axis direction, and directions intersecting the Z-axis direction are defined as an X-axis direction and a Y-axis direction as in FIG. 6A.

The substrate 30s has the communication unit 45, a microcomputer circuit 50, a power supply circuit 51, and a power supply unit 52 on a surface having a microcomputer circuit 50.

The communication unit 45 can transmit information regarding a detection result of the detector 30 to an external device. The detection result includes, for example, information (data) related to a concentration of the detection target. The transmission may be performed, for example, in at least one of a wired or a wireless manner.

The microcomputer circuit 50 performs control of data communication, control of a sensor circuit, and on/off control of the second electrochemical element 10.

The microcomputer circuit 50 includes, as a software configuration that functions by executing a built-in control program, a dehumidifying operation that operates the second electrochemical element 10 from a change in a detection device in the detector 30 to maintain the humidity in a target range.

The power supply circuit 51 supplies an appropriate voltage to the microcomputer circuit and the sensor circuit.

The power supply unit 52 is a portion connected to a battery serving as a power supply.

The substrate 30s has a sensor circuit on the surface opposite to the surface on which the microcomputer circuit 50 is provided. On the surface of the substrate 30s having the sensor circuit, the substrate 30s has the detector 30, the first electrochemical element 46, an AD conversion circuit 53, a step-up circuit 54 (voltage booster circuit), and a step-down circuit 55 (voltage drop circuit).

For the detector 30, for example, when humidity in the environment around the detector 30 changes, a detection value of a detection target in the detector 30 is affected. By using this detection value to maintain the humidity in the environment around the detector 30 in a target range, it is possible to suppress deterioration or failure of the sensing function due to exposure of the sensor and the sensor circuit for a long time in a high-humidity environment. This makes it possible to detect oxygen and hydrogen with higher accuracy.

The detector 30 can detect at least one selected from a group consisting of hydrogen, oxygen, and volatile organic compounds (VOCs).

The AD conversion circuit 53 is a circuit that converts a voltage and a capacitance analog signal into a voltage digital signal.

The first electrochemical element 46 is a sensor that measures humidity and temperature, and monitors humidity and temperature in a space inside the housing. The first electrochemical element 46 may also be a pressure sensor.

The step-up circuit 54 and the step-down circuit 55 are used to adjust power to be supplied to the sensor.

Although FIGS. 6A to 6C illustrate the example in which the microcomputer circuit is provided on one surface of the substrate and the sensor circuit is provided on the other surface on the opposite side, the substrate on which the microcomputer circuit 50 is provided and the substrate on which the sensor circuit is provided may be separately prepared. In such a case as described above, for example, by connecting each substrate with a cable connector or the like, a control signal from the microcomputer circuit 50 is transmitted to the sensor circuit, and data acquired from the sensor circuit is transmitted to the microcomputer circuit 50.

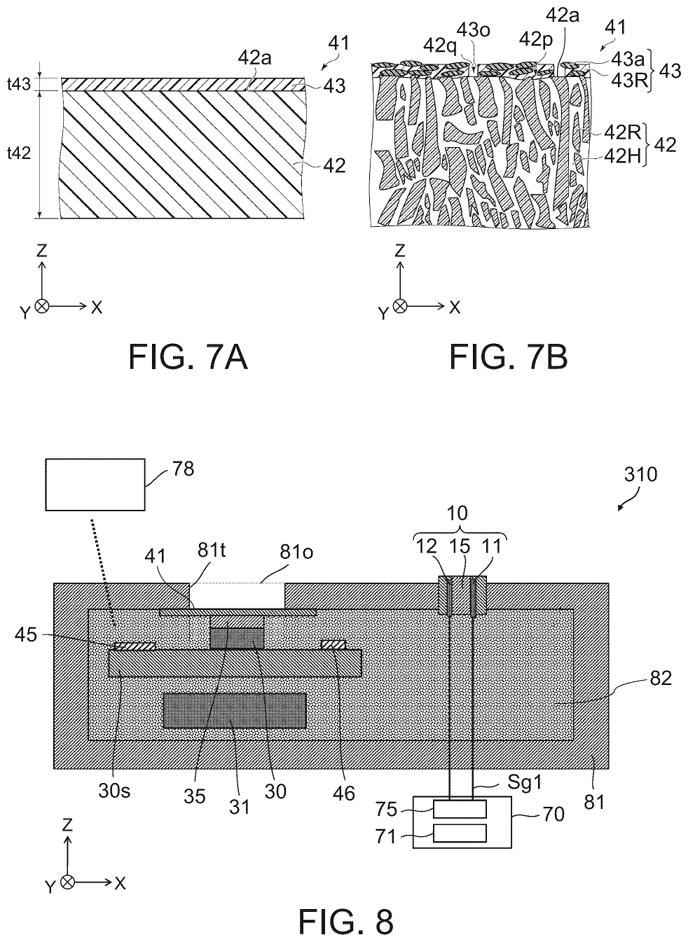

Next, the first film that can be included in the electrochemical device according to the second embodiment will be described below with reference to FIGS. 7A and 7B.

FIGS. 7A and 7B are schematic cross-sectional views illustrating the first film that can be included in the electrochemical device according to the second embodiment. As illustrated in FIG. 7A, the first film 41 may include a first layer 42 and a second layer 43. A direction from the first layer 42 to the second layer 43 is defined as the Z-axis direction. One direction perpendicular to the Z-axis direction is defined as the X-axis direction. A direction perpendicular to the Z-axis direction and the X-axis direction is defined as the Y-axis direction. The first layer 42 and the second layer 43 extend along the X-Y plane.

As illustrated in FIG. 7B, the first layer 42 includes a first resin 42R. The first resin 42R is provided with a plurality of holes 42H. The first layer 42 is, for example, a porous layer.

The first layer 42 includes a first surface 42a. The first surface 42a is a surface facing the second layer 43. At least a part of the plurality of holes 42H reaches the first surface 42a.

The second layer 43 is provided on the first surface 42a of the first layer 42. The second layer 43 includes a second resin 43R. The second resin 43R blocks at least a part of the plurality of holes 42H reaching the first surface 42a. The second layer 43 is, for example, an ineffective void layer (independent void layer). A part of the second resin 43R may be provided in a portion close to the surface of the hole 42H.

In the embodiment, the porous first layer 42 and the second resin 43R (second layer 43) that blocks a part of the plurality of holes 42H of the first layer 42 are provided. For example, another part of the plurality of holes 42H of the first layer 42 is not blocked by the second resin 43R.

The hole 42H not blocked by the second resin 43R allows a target gas to pass through the first film 41. The target gas is, for example, hydrogen. On the other hand, since a part of the plurality of holes 42H is blocked by the second resin 43R, a non-target substance does not pass through the first film 41. Non-target substances include, for example, liquids (such as water and oil). According to the embodiment, the target gas can efficiently permeate.

Due to the first film 41 that can be included in the electrochemical device according to the embodiment, for example, high water repellency can be obtained. For example, high air permeability can be obtained. For example, high chemical resistance can be obtained. For example, high corrosion resistance can be obtained. For example, high dustproofness can be obtained. For example, entry of water or oil is suppressed. For example, high reliability can be obtained.

In the embodiment, the first resin 42R preferably contains a fluorine compound. The first resin 42R contains, for example, polytetrafluoroethylene (PTFE). Stable permeability can be obtained. Permeation of water or the like can be effectively suppressed.

The second resin 43R contains, for example, an acrylic resin. As a result, at least a part of the plurality of holes 42H is stably blocked. For example, permeation of a non-target substance can be stably suppressed.

The thickness of the first layer 42 is defined as a first thickness t42. The thickness of the second layer 43 is defined as a second thickness t43. These thicknesses are lengths along the Z-axis direction. In the embodiment, for example, the first thickness t42 is thicker than the second thickness t43. In one example, the first thickness t42 is at least twice as thick as the second thickness t43. For example, the first thickness t42 may be 10,000 times or less than the second thickness t43. For example, permeability of the target gas and suppression of permeation of a non-target substance can be appropriately obtained.

The first thickness t42 of the first layer 42 is, for example, 10 μm or more and 5,000 μm or less. The first thickness t42 may be, for example, 1,000 μm or less. The second thickness t43 of the second layer 43 is, for example, 0.1 μm or more and 100 μm or less. When a part of the second resin 43R is provided in the portion close to the surface of the hole 42H, the thickness of the second resin 43R provided in the portion close to the surface of the hole 42H may be, for example, 0.1 μm or more and 5 μm or less.

The second resin 43R covers at least a part of the first surface 42a. As illustrated in FIG. 7B, the first surface 42a includes a first region 42p in which the second resin 43R is provided and a second region 42q in which the second resin 43R is not provided. A ratio of the area of the first region 42p to the area of the second region 42q is 0.01 or more and 100 or less.

For example, the second resin 43R may include an opening 430. The second region 42q where the second resin 43R is not provided corresponds to the opening 430. An opening ratio may be, for example, 1% or more and 99% or less.

As illustrated in FIG. 7B, in this example, the second layer 43 further includes a plurality of first solid pieces 43a. The plurality of first solid pieces 43a are fixed by the second resin 43R. The plurality of first solid pieces 43a contain, for example, at least one of a metal, a metal oxide, and a metal nitride. For example, the plurality of first solid pieces 43a contain Fe, Cr, and Ni. The plurality of first solid pieces 43a contain, for example, steel use stainless (SUS). For example, high corrosion resistivity can be obtained. The plurality of first solid pieces 43a may contain, for example, titanium oxide.

An average size (length) of one of the plurality of first solid pieces 43a is, for example, 0.1 μm or more and 10 μm or less.

The sensor according to the second embodiment is the sensor including the electrochemical device described in the first embodiment and the detector provided inside the housing. Since the sensor according to the second embodiment includes the electrochemical device described in the first embodiment, it is possible to realize a sensor with improved performance.

### Third Embodiment

According to a third embodiment, there is provided a sensor system including the sensor according to the second embodiment and a processing device, in which the sensor includes a communication unit, and the processing device can process information based on a signal obtained from the communication unit.

FIG. 8 is a schematic cross-sectional view illustrating the sensor according to the third embodiment. As illustrated in FIG. 8, a sensor system 310 according to the embodiment includes the sensor 210 according to the second embodiment and a processing device 78. The processing device 78 may include, for example, a computer. The sensor 210 includes the communication unit 45. The processing device 78 can process information based on the signal obtained from the communication unit 45. The signal obtained from the communication unit 45 may include, for example, information (data) related to the detection result of the detector 30.

The processing of the information (detection result) in the processing device 78 may include, for example, saving of the information (detection result). The processing of the information (detection result) may include, for example, a comparison between the information (detection result) and a reference value. The processing device 78 may output an alert or the like according to a result of the comparison. The processing of the information (detection result) may include, for example, an arbitrary calculation related to the information (detection result). The calculation may include, for example, derivation of a maximum value, or the like, or derivation of an average value, or the like.

The sensor system according to the third embodiment includes the sensor according to the second embodiment and the processing device, in which the sensor includes the communication unit, and the processing device can process information based on the signal obtained from the communication unit.

### Examples

Hereinafter, examples of results of experiments performed by the inventors will be described. Table 1 shows the rate of increase in humidity detected by the humidity sensor inside the housing when the electrochemical device having the porous structure inside the housing is placed under a humidity atmosphere of 90% relative humidity (RH). A porous PTFE film was used as the first film. The numerical values of the examples are described assuming that the rate of increase in humidity when the electrochemical device does not include the porous structure is 1.

TABLE 1

| Type | | Humidity increase |
|---|---|---|
| of porous | Filling | rate (where none |
| structure | rate (%) | is 1) |
| None | 0 | 1 |
| Zeolite | 100 | 0.2 |
| Zeolite | 90 | 0.25 |
| Zeolite | 50 | 0.4 |
| Zeolite | 30 | 0.8 |
| Activated carbon | 50 | 0.5 |
| Activated carbon | 20 | 0.9 |
| Silica gel | 50 | 0.5 |

From Table 1, it can be seen that the humidity increase rate detected by the humidity sensor can be reduced by the porous structure occupying at least a part of the inside of the housing as compared with the case where there is no porous structure inside the housing. Therefore, the electrochemical device includes the porous structure that occupies at least a part of the inside of the housing, and thus, it is possible to suppress exposure of the sensor and the sensor circuit to water vapor for a long time, and to suppress deterioration of a sensing function of the sensor and a failure of the sensor circuit included in the sensor.

In the above-described embodiments, the opening is provided in the housing, a porous film (first film 41) is installed in the opening, and the detector (30) is disposed so as to be in contact with the porous film. The humidity of the detector depends on the humidity inside the housing, and when the humidity inside the housing is low, the humidity of the detector is also low. Under a high humidity environment, moisture is permeated into the housing through the detector. Therefore, if the high humidity environment continues for a long time, the internal humidity increases and the humidity of the detector also increases. When the humidity exceeds a certain humidity, it is necessary to operate the dehumidifier (second electrochemical element 10) to suppress an increase in humidity of the detector. However, since the dehumidifier is driven by a battery and cannot be driven for a long time, it is desired to reduce the operating time of the dehumidifier.

Furthermore, when the sensor substrate (substrate 30s) and the housing are made of resin, the inside of the housing and the sensor substrate absorb moisture. Under a high humidity environment, moisture is permeated into the housing through the detector. Therefore, if the high temperature and high humidity environment continues for a long time, the inner wall of the housing and the sensor substrate absorb moisture. In a case where the ambient temperature rises, moisture is released from the housing wall and the sensor substrate, so that humidity in the housing increases.

On the other hand, in order to suppress an increase in humidity of the detector, a moisture permeation rate inside the housing may be suppressed. The amount of absolute humidity transmitted through the detector per unit time inside the housing is constant, and the volume of the housing may be increased to suppress the moisture permeation rate. However, it is not realistic to make the housing excessively large. By providing the porous structure inside the housing as in the above-described embodiment, the effective volume can be increased. By covering the sensor substrate and the housing inner wall with a porous material, the humidity released from the housing inner wall and the sensor substrate can be absorbed to suppress the moisture permeation rate.

The filling of the zeolite shown in the table may be performed by filling the housing with granular PE resin pellets (polyethylene resin pellets) containing zeolite.

In the above-described embodiments, the detector is, for example, a gas detector. The detector can detect, for example, at least one selected from the group consisting of hydrogen, oxygen, and VOC. The first electrochemical element is, for example, a sensor that measures humidity and/or temperature. The first electrochemical element may also be a pressure sensor. The second electrochemical element can perform at least one of, for example, dehumidification, humidification, ozone generation, oxygen generation, oxygen removal, and hydrogen generation. The second electrochemical element is, for example, a dehumidifying element.

The filling rate of the porous structure is preferably 10% or more and 100% or less. At this time, various components in the housing may be removed from the volume of the housing.

Although some embodiments of the present invention have been described, these embodiments have been presented as examples, and are not intended to limit the scope of the invention. These novel embodiments can be implemented in various other forms, and various omissions, substitutions, and changes can be made without departing from the gist of the invention. These embodiments and modifications thereof are included in the scope and gist of the invention, and are included in the invention described in the scope of the claims and the equivalent scope thereof.

Hereinafter, one of the aspects according to the embodiment will be additionally described.

[1]

An electrochemical device including:
a housing;
a first electrochemical element present inside the housing; and
a porous structure occupying at least a part of the inside of the housing.
   [2]

The electrochemical device according to [1], in which the porous structure covers at least a part of an inner wall of the housing.

[3]

The electrochemical device according to [1] or [2], further including:
a substrate; and
a first battery,
in which the first electrochemical element is provided on the substrate, and
the first battery is provided on a surface on the substrate opposite to a surface on which the first electrochemical element is provided.
   [4]

The electrochemical device according to [3], in which the porous structure is present between the substrate and the first battery.

[5]

The electrochemical device according to any one of [1] to [4], in which the housing has a second electrochemical element.

[6]

The electrochemical device according to [5], in which the porous structure provided inside the housing covers at least a part of the second electrochemical element.

[7]

The electrochemical device according to any one of [1] to [6], in which the porous structure is at least one selected from the group consisting of zeolite, silica gel, activated alumina, activated carbon, and a porous polymer.

[8]

The electrochemical device according to any one of [1] to [7], in which a filling rate of the porous structure inside the housing excluding the first electrochemical element provided inside the housing is 10% or more and 100% or less.

[9]

A sensor including:
the electrochemical device according to any one of [1] to [8]; and
a detector provided inside the housing.
   [10]

A sensor system including:
the sensor according to [9]; and
a processing device,
in which the sensor includes a communication unit,
and the processing device is capable of processing information based on a signal obtained from the communication unit.

What is claimed is:

1. An electrochemical device comprising:
a housing;
a first electrochemical element present inside the housing; and
a porous structure occupying at least a part of the inside of the housing.

2. The electrochemical device according to claim 1, wherein the porous structure covers at least a part of an inner wall of the housing.

3. The electrochemical device according to claim 1, further comprising:
a substrate; and
a first battery,
wherein the first electrochemical element is provided on the substrate, and
the first battery is provided on a surface on the substrate opposite to a surface on which the first electrochemical element is provided.

4. The electrochemical device according to claim 3, wherein the porous structure is present between the substrate and the first battery.

5. The electrochemical device according to claim 1, wherein the housing has a second electrochemical element.

6. The electrochemical device according to claim 5, wherein the porous structure provided inside the housing covers at least a part of the second electrochemical element.

7. The electrochemical device according to claim 1, wherein the porous structure is at least one selected from the group consisting of zeolite, silica gel, activated alumina, activated carbon, and a porous polymer.

8. The electrochemical device according to claim 1, wherein a filling rate of the porous structure inside the housing excluding the first electrochemical element provided inside the housing is 10% or more and 100% or less.

9. A sensor comprising:
the electrochemical device according to claim 1; and
a detector provided inside the housing.

10. A sensor system comprising:
the sensor according to claim 9; and
a processing device,
wherein the sensor includes a communication unit,
and the processing device is capable of processing information based on a signal obtained from the communication unit.

11. A sensor comprising:
a housing;
a detector provided inside the housing; and
a porous structure that occupies at least a part of the inside of the housing.

12. The sensor according to claim 11, wherein the porous structure covers at least a part of an inner wall of the housing.

13. The sensor according to claim 11, further comprising:
a substrate;
a first electrochemical element present inside the housing; and
a first battery,
wherein the first electrochemical element is provided on the substrate, and
the first battery is provided on a surface on the substrate opposite to a surface on which the first electrochemical element is provided.

14. The sensor according to claim 13, wherein the porous structure is present between the substrate and the first battery.

15. The sensor according to claim 11, wherein the housing has a second electrochemical element.

16. The sensor according to claim 15, wherein the porous structure provided inside the housing covers at least a part of the second electrochemical element.

17. The sensor according to claim 11, wherein the porous structure is at least one selected from the group consisting of zeolite, silica gel, activated alumina, activated carbon, and a porous polymer.

18. The sensor according to claim 11, wherein a filling rate of the porous structure inside the housing excluding the detector provided inside the housing is 10% or more and 100% or less.

19. A sensor system comprising:
the sensor according to claim 11; and
a processing device,
wherein the sensor includes a communication unit, and
the processing device is capable of processing information based on a signal obtained from the communication unit.
